(19) United States
(12) Patent Application Publication
(10) Pub. No.: US 2011/0270395 A1
(43) Pub. Date: Nov. 3, 2011

(54) DEVICE AND METHOD FOR DELIVERING RADIATION IN SELECTED DIRECTIONS

(75) Inventors: Jonathan E. Blackwell, Arlington, TN (US); Keith E. Miller, Germantown, TN (US)

(73) Assignee: Warsaw Orthopedic, Inc., Warsaw, IN (US)

(21) Appl. No.: 12/769,346

(22) Filed: Apr. 28, 2010

(51) Int. Cl. A61F 2/44; A61N 5/00

(52) U.S. Cl. 623/17.11; 600/3

(57) ABSTRACT

Embodiments of the invention include a device for supplementing or replacing a spinal structure and therapeutically delivering radiation to tissue. Some embodiments include a radiation source and a combination of members surrounding the radiation source that move relative to one another to permit or restrict emission of radiation from the device.

### FIELD OF THE INVENTION

The present invention relates generally to the field of supplementing or replacing orthopedic structures, and more particularly relates to supplementing or replacing spinal structures with an implant capable of selectively permitting radiation to be therapeutically delivered to tissue within or near the spinal structures.

### BACKGROUND

In some circumstances, an implant is used to supplement or replace an orthopedic structure, like a spinal structure. Such an implant may be used to respond to a spinal pathology, as part of a cancer treatment, or for any effective purpose or combination of purposes. For example and without limitation, an implant may be an interbody spinal implant, a vertebral body replacement implant Implants classified as vertebral body replacement implants may include implants used in association with corpectomy or vertebrectomy procedures to stabilize spinal structures. Removal, or excision, of a vertebra may be referred to as a vertebrectomy. Excision of a generally anterior portion, or vertebral body, of the vertebra may be referred to as a corpectomy. If only a portion of a vertebral body and adjacent discs are removed and replaced, the procedure may be called a hemi-vertebrectomy. An implant used to stabilize, supplement, or replace a spinal structure may also serve as a platform to assist with the delivery of radiation treatment toward adjacent tissues suspected of including or known to include one or more of cancerous cells and tumors. An improved device may include the capability to selectively permit or restrict emitting of radiation. Some improved devices may be capable of directing treatment in one or more specified directions.

### SUMMARY

One embodiment of the invention is a device for supplementing or replacing a spinal structure and therapeutically delivering radiation to tissue within or near the spinal structure. The device may include a spinal implant core configured to be placed between a first vertebra and a second vertebra to supplement or replace at least a portion of the spinal structure. The spinal implant core may have at least one wall with a length between a first end and a second end. Spinal implant core embodiments include one or more radiolucent areas and one or more areas comprising a material that substantially blocks the transmission of radiation. The device may include a radiation source located within the spinal implant core and configured to deliver radiation. The device may also include a shield coupled to the spinal implant core that is movable relative to the spinal implant core. The shield has a length between a first end and a second end. The shield may include one or more radiolucent areas that may be aligned with the one or more radiolucent areas of the spinal implant core to selectively permit radiation to be therapeutically delivered to tissue within or near the spinal structure.

An embodiment of the invention is a device for supplementing or replacing a spinal structure between a first vertebra and a second vertebra and for therapeutically delivering radiation to tissue within or near the spinal structure. The device includes a first tubular member having at least one wall with a length between a first end and a second end. The first end may contact the first vertebra and the second end may contact the second vertebra. Embodiments of the first tubular member include one or more radiolucent areas and one or more areas comprising a material that substantially blocks the transmission of radiation. The device may also include a second tubular member coupled to the first tubular member and having at least one wall with a length between a first end and a second end. The second tubular member may include one or more radiolucent areas and one or more areas comprising a material that substantially blocks the transmission of radiation. The second tubular member may be movable relative to the first member to control alignment of radiolucent areas of the first tubular member and radiolucent areas of the second tubular member. The first tubular member and the second tubular member may be configured to receive a radiation source.

Another embodiment of the invention is a method of irradiating cells in or near a spinal structure. The method may include providing a spinal implant core in which a radiation source is substantially enclosed. The spinal implant core may be configured to replace at least a portion of the spinal structure, and the spinal implant core may include one or more radiolucent areas and one or more areas comprising a material that substantially blocks the transmission of radiation. A shield coupled to the spinal implant core that is movable relative to the spinal implant core may also be provided. Embodiments of the shield include one or more radiolucent areas and one or more areas comprising a material that substantially blocks the transmission of radiation. The method may also include the act of implanting the spinal implant core, the radiation source, and the shield into a spinal structure between vertebrae to reinforce the spinal structure. The method may include aligning at least one radiolucent area of the spinal implant core with at least one radiolucent area of the shield to allow radiation to be therapeutically delivered to cells in or near the spinal structure, and the method may include aligning each radiolucent area of the spinal implant core with an area of the shield that comprises a material that substantially blocks the transmission of radiation and aligning each radiolucent area of the shield with an area of the spinal implant core that comprises a material that substantially blocks the transmission of radiation such that radiation is not therapeutically delivered to cells in or near the spinal structure.

### BRIEF DESCRIPTION OF THE DRAWINGS

FIG. 1 is a perspective view of an embodiment of a device for supplementing or replacing a spinal structure and therapeutically delivering radiation to tissue implanted between vertebrae.

FIG. 2 is a cross-sectional view of the device of FIG. 1.

FIG. 3 is a cross-sectional view of the device of FIG. 2 in a different state of device component movement.

FIG. 4 is a perspective view of a portion of the device of FIG. 1.

FIG. 5 is a cross-sectional view of the portion of the device of FIG. 4.

FIGS. 6A-6E are cross-sectional views of an embodiment of a device for supplementing of replacing a spinal structure and therapeutically delivering radiation to tissue in progressive states of device component movement.

FIG. 7 is a plan view of an embodiment of a device for supplementing or replacing a spinal structure and therapeutically delivering radiation to tissue

### DETAILED DESCRIPTION

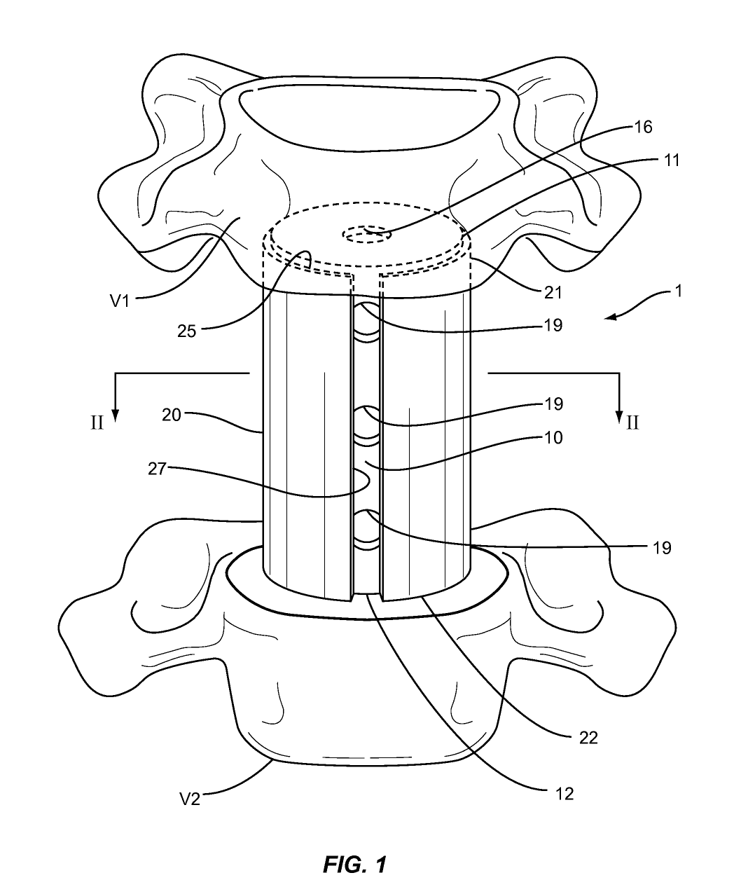

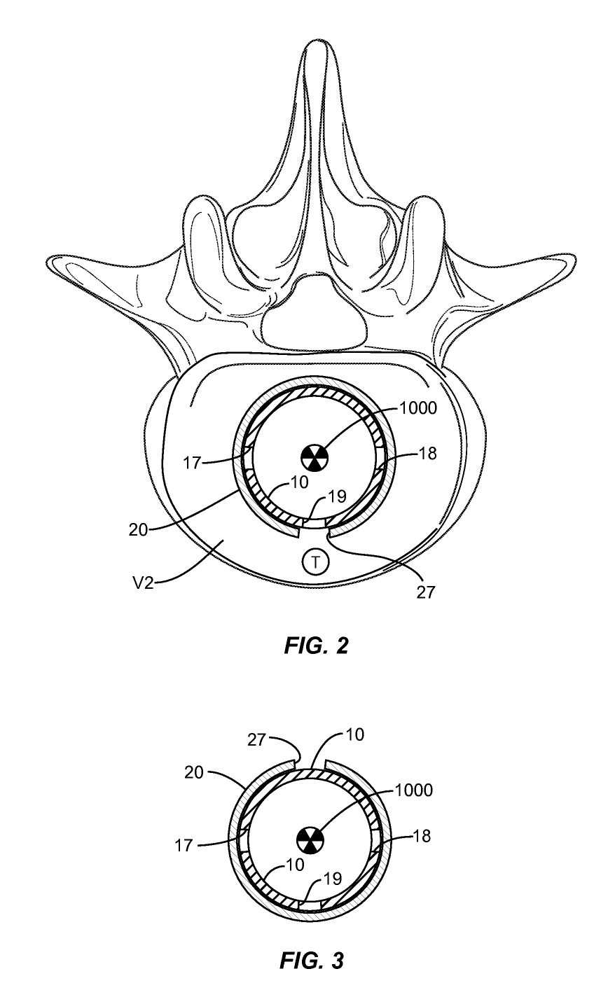

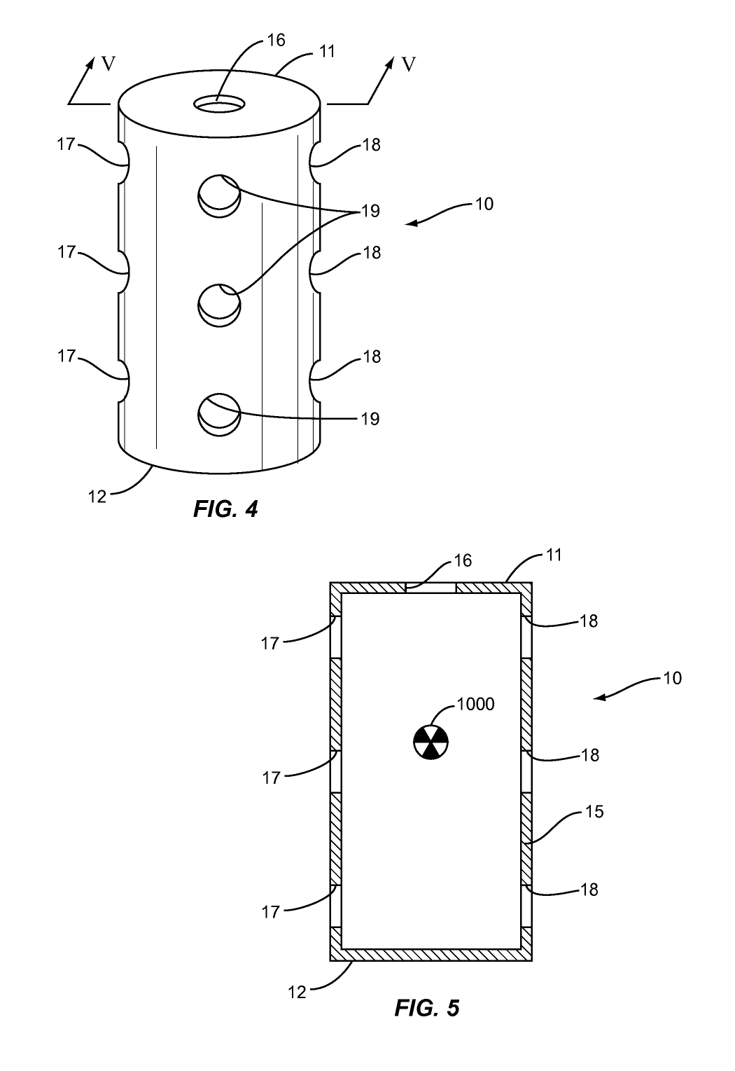

An embodiment of a device 1 for supplementing or replacing one or more spinal structures and therapeutically delivering radiation to tissue within or near the spinal structures is illustrated in FIGS. 1-5. The device 1 illustrated is a vertebral body replacement implant. However, in other embodiments, the device may be any implant that may be used in a space between two vertebrae, such as the illustrated first vertebra V1 and the second vertebra V2. The lateral periphery of the device 1 is substantially round in cross-section, as particularly illustrated in FIGS. 2 and 3. Other embodiments may have a periphery that is substantially the shape of an oval, kidney, triangle, rectangle, square, any polygonal or curved shape, or any combination of shapes. In some embodiments, a vertebral body replacement type device may be configured to expand from a first height of a second taller height. The device 1 or any of its component parts may be made from any biocompatible material.

The device 1 or any of its component parts may have selected areas that are radiolucent and selected areas that include, in whole or in part, one or more materials that substantially block transmission of radiation. Materials that substantially block the transmission of radiation include, but are not limited to, cobalt chrome, titanium, stainless steel, tantalum, niobium, gold, lead, barium, bismuth, tin, and tungsten. A radiation blocking material may be applied to the inside or outside or be encapsulated within a component so that only certain of the materials are in direct communication with tissues or fluids of a patient. A radiation blocking material may be applied to or integrated with a component by any effective mechanism, including but not limited to, chemically bonding, an intervening adhesive, welding, melting, press fitting, ion deposition, or mechanically locking. As used herein, the term “blocking the transmission of radiation” and similar terms mean that a material, composite, or component blocks the passage of therapeutically effective amounts of radiation from a radiation source. The blocking of radiation may not be complete such that there is no measurable amount of radiation allowed through a component.

Radiolucent materials used may include polyetheretherketone (PEEK) or a PEEK composite, some metal alloys, various other polymers and composites, and bone or bone-based materials. For example and without limitation, bone or bone-based materials may include one or more of allograft, autograft, xenograft, and demineralized bone. Additionally, areas of radiolucency, as specified herein, may be areas where openings have been created through material that would otherwise substantially block the transmission of radiation.

As shown in FIG. 1, the device 1 includes a spinal implant core 10 in the form of a first tubular member placed between the first vertebra V1 and the second vertebra V2. The spinal implant core 10 serves as a component in support of the illustrated spinal structure. The spinal implant core 10 illustrated in FIGS. 1-5 includes one wall 15 (FIG. 5) formed into a substantially round cross-section tubular member. Other embodiments may include two or more walls forming various shapes, including polygonal shapes and combination shapes. The spinal implant core 10 shown includes a length between a first end 11 and a second end 12. The illustrated spinal implant core 10 includes radiolucent areas in the form of openings 17, 18, 19 formed through the wall 15. A top opening 16 is illustrated in FIGS. 1, 4, and 5 providing a radiolucent area. In other embodiments, radiolucent areas may be any material or composite as described above that allows a therapeutically effective amount of radiation to penetrate through the spinal implant core 10. In some embodiments, no top opening is provided, or a selectable top or bottom opening is provided. Most other portions of the spinal implant core 10 shown in the illustrated embodiment are areas that include materials that substantially block the transmission of radiation.

The device 1 may also include a radiation source in the form of a radiation emitting device 1000, as illustrated in FIGS. 2, 3, and 5, configured to deliver radiation in some embodiments. The radiation emitting device 1000 is shown within the spinal implant core 10. In other embodiments, a radiation emitting device may be located at any effective location in or on a device. Radiation may be emitted in any or all directions from the radiation emitting device 1000 in various embodiments. Although the radiation emitting device 1000 is shown in approximately the middle of the spinal implant core 10, the radiation emitting device 1000 may be located anywhere or everywhere within the spinal implant core 10 or the device 1. The radiation emitting device may be of any size and located to approximately distribute radiation evenly out of the openings 17, 18, 19, and the top opening 16, or any other configuration of openings. The direction and pattern of radiation transmission may be altered by the shape, orientation, and placement of the radiation emitting device 1000.

The radiation emitting device 1000 may include any therapeutically effective radiation source. Suitable radiation sources for use in the radiation emitting device 1000 of some embodiments include both solids and liquids. By way of non-limiting example, the radiation source may be a radionuclide, such as I-125, I-131, Yb-169, Ir-192 or other radionuclides that emit photons, beta particles, gamma radiation, or other therapeutic energy or substances. The radioactive material may also be a fluid made from any solution of radionuclide(s), e.g., a solution of I-125 or I-131, or a radioactive mixture may be produced using a slurry of a suitable fluid containing small particles of solid radionuclides, such as Au-198, Y-90. Radionuclides may also be delivered in a gel. One radioactive material useful in some embodiments is Iotrex®, a nontoxic, water soluble, nonpyrogenic solution containing sodium 3-(125I)iodo-4-hydroxybenzenesulfonate (125I-HBS), available from Proxima Therapeutics, Inc. of Alpharetta, Ga. Radioactive micro spheres of the type available from the 3M Company of St. Paul, Minn., may also be used. A radioactive source may be preloaded into a device 1 at the time of manufacture, at some other time prior to a surgical procedure, or loaded after the device 1 has been implanted. By way of further non-limiting example, one or more solid radioactive micro spheres may be inserted through a catheter on a wire and into a device.

A shield 20, in the form of a second tubular member, is shown coupled to the spinal implant core 10 in FIGS. 1-3 and is movable relative to the spinal implant core 10. The shield 20 illustrated has a length between a first end 21 and a second end 22. The shield 20 illustrated includes one wall 25 (FIG. 1) formed into a substantially round cross-section tubular member. An opening 27, in the form of a slot, is shown in the one wall 25 of the shield 20. Although the shield 20 is cut from the first end 21 to the second end 22 to form the opening 27, it may still be designated as a tubular member, as used herein. In other embodiments, openings in the wall 25 may be smaller slots or holes of any effective configuration. Other embodiments may include two or more walls forming various polygonal shapes and combination shapes. The illustrated shield 20 includes radiolucent areas in the form of the opening 27 formed through the wall 25. Additionally, the top and the bottom of the shield 20 are open and provide radiolucent areas. In other embodiments, radiolucent areas may be any material or composite as described above that allows a therapeutically effective amount of radiation to penetrate through the shield 20. In some embodiments, no openings on top or bottom of a shield are provided, or a selectable top or bottom opening is provided. Most other portions of the shield 20 shown in the illustrated embodiment are areas that include materials that substantially block the transmission of radiation.

The shield 20 shown is movable relative to the spinal implant core 10 about a common longitudinal axis. The shield 20 may rotate about the spinal implant core 10, as shown in the transition of positions between FIGS. 2 and 3. In FIG. 2, a radiolucent area of the spinal implant core 10, as embodied in openings 19, is aligned with the radiolucent areas of the shield 20, as embodied in the opening 27, to selectively permit radiation to be therapeutically delivered to tissue within or near the spinal structure. Radiation from the radiation emitting device 1000 is permitted to travel through the openings 19 and 27 and deliver therapeutic dosages to a treatment site “T” depicted in FIG. 2. In FIG. 3, where none of the openings 17, 18, 19 or other radiolucent areas are aligned with the opening 27 or other radiolucent areas in the wall 25 of the shield 20, radiation is not permitted to be delivered laterally from the device 1. In other embodiments, where, for example, a shield is of a lesser length than a spinal implant core and radiolucent openings in the shield and the spinal implant core are offset along the longitudinal axis of the device, radiation may be permitted to be delivered from the device by moving the shield relative to the spinal implant core along the longitudinal axis of the device. Any combination of rotational or linear movements may be used in various embodiments to align and misalign radiolucent areas of the components.

Movement between a spinal implant core and a shield may be driven by any effective actuator, and may include one or more drive mechanisms and signal devices. Drive components may be housed within the spinal implant core, between the spinal implant core and the shield, outside of the shield, in other effective locations, or in any combination of these locations. Example drive mechanisms include, but are not limited to, micromotors, magnetic drives, ratchet drives, piezoelectric drives, hydraulic actuators, other effective mechanisms, and combinations of these drives. Signals to drive these mechanisms may be provided by wired or wireless transmission, physical attachment, hydraulic actuation, radio signal, or any other effective signal or mechanism for the drive mechanism selected.

While the illustrated shield 20 is coupled to the spinal implant core 10 on an outside surface of the spinal implant core 10 wall 15, other embodiments of the shield may be coupled on an inside surface of a spinal implant core. The spinal implant core 10 is the portion of the device 1 that makes primary contact with the vertebrae V1, V2 in the illustrated embodiment. The shield 20 in this and other embodiments may make limited contact with the vertebrae V1, V2, or may not be permitted to contact the vertebrae V1, V2. In some embodiments, a shield may make primary contact with the vertebrae V1, V2, and a spinal implant core may be allowed to move relative to the shield to align or mis align radiolucent areas with limited or not contact with vertebrae.

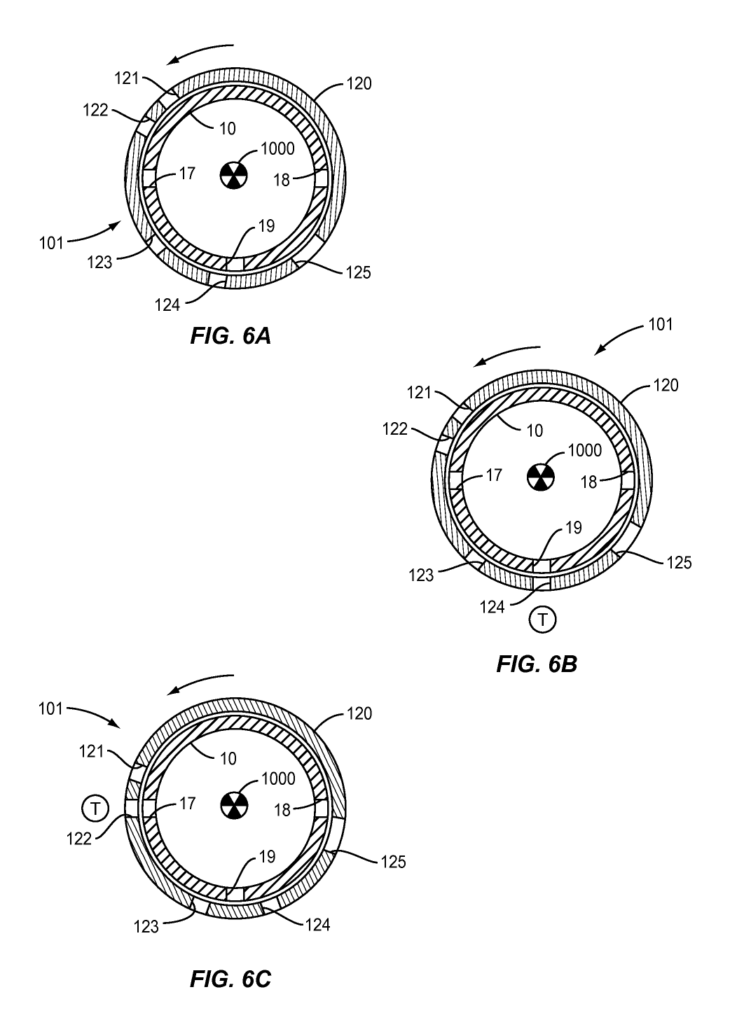

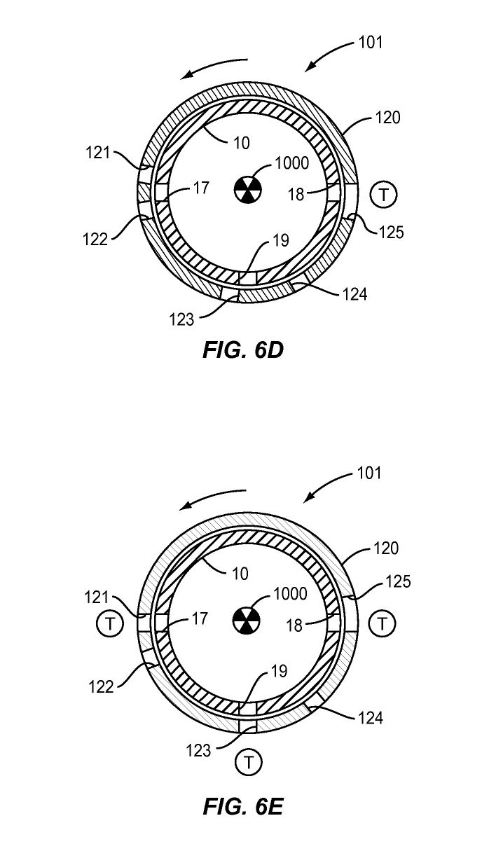

An alternative embodiment of a device for supplementing or replacing one or more spinal structures and therapeutically delivering radiation to tissue within or near the spinal structures is a device 101, illustrated in FIGS. 6A-6E. The device 101 is generally similar to the device 1, but includes a different configuration of radiolucent areas in a shield 120. Five states of movement between the spinal implant core 10 and the shield 120 are shown in FIGS. 6A-6E. As illustrated, five different radiolucent areas, embodied in five openings 121-125, may be aligned with one or more of the radiolucent areas in the spinal implant core 10, embodied in one or more of the three openings 17-19. Any of the openings may be round, slotted, any polygonal shape, or any other shape effective to direct radiation in any desired direction for various embodiments of the device. In the illustrated embodiment, the opening 125 is wider than other openings so that the opening 125 will align with the opening 18 consecutively in the states depicted in both FIGS. 6D and 6E. Each of the different states of rotation of the shield 120 relative to the spinal implant core 10 provides for a different alignment of radiolucent areas, and therefore, a different emitted pattern of radiation. Any effective number of radiolucent areas in a spinal implant core and a shield may be used. In the illustrated embodiment, movement between the spinal implant core 10 and the shield 120 is by rotation about a common longitudinal axis and is graphically represented by a counterclockwise arrow along the periphery of each shield 120. Rotation about a different axis or linear motion may also be employed in some embodiments. As noted above, a shield component of a device may also be located inside of a spinal implant core.

Referring to the specific states depicted in FIGS. 6A-6E, in FIG. 6A, no radiation is emitted laterally from the device 101 because none of the radiolucent areas of the spinal implant core 10 align with any of the radiolucent areas of the shield 120. In FIG. 6B, the shield 120 has been advanced relative to the spinal implant core 10 in a counterclockwise direction so that the opening 124 is aligned with the opening 19. Radiation from the radiation emitting device 1000 is permitted to travel through the openings 19 and 124 and deliver therapeutic dosages to a treatment site “T” depicted in FIG. 6B. In FIG. 6C, the shield 120 has been advanced relative to the spinal implant core 10 in a counterclockwise direction so that the opening 122 is aligned with the opening 17. Radiation from the radiation emitting device 1000 is permitted to travel through the openings 17 and 122 and deliver therapeutic dosages to a treatment site “T” depicted in FIG. 6C. In FIG. 6D, the shield 120 has been advanced relative to the spinal implant core 10 in a counterclockwise direction so that the opening 125 is aligned with the opening 18. Radiation from the radiation emitting device 1000 is permitted to travel through the openings 18 and 125 and deliver therapeutic dosages to a treatment site “T” depicted in FIG. 6D. In FIG. 6E, the shield 120 has been advanced relative to the spinal implant core 10 in a counterclockwise direction so that the openings 121, 123, and 125 are aligned respectively with the openings 17, 19, and 20. Radiation from the radiation emitting device 1000 is permitted to travel through the opening pairs 17/121, 19/123, and 18/125 and deliver therapeutic dosages to the three treatment sites “T” depicted in FIG. 6E. An additional incremental counterclockwise rotation of the shield 120 relative to the spinal implant core 10 would result in a state where no radiation is emitted laterally from the device 101 because none of the radiolucent areas of the spinal implant core would 10 align with any of the radiolucent areas of the shield 120. The device 101 permits radiation to be emitted in each of the directions of the openings 17-19 with less relative movement between the spinal implant core 10 and the shield 120, which may be advantageous in some circumstances.

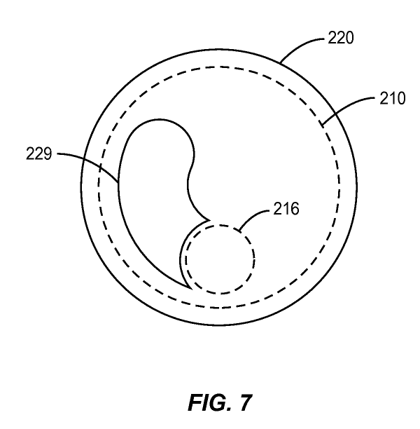

FIG. 7 illustrates an alternative embodiment for a top or bottom of a device. As shown, a spinal implant core 210 with an opening 216 is coupled within a shield 220. The opening 216 is offset from the common rotational axis of the spinal implant core 210 and the shield 220. The shield 220 includes an opening 229 that is on a path to align with the opening 216 when relative rotation of the spinal implant core 210 and the shield 220 is accomplished. When the openings 216 and 229 are aligned, radiation from a radiation source within the spinal implant core 210 would be allowed to be emitted. Although depicted as openings 216 and 229, these openings may also be areas of radiolucency though solid materials as described above. Openings in the top or bottom of a shield or a spinal implant core may be of any effective shape and are not necessarily shaped as shown in FIG. 7.

Any of the devices described above may be filled in whole or in part with an osteogenic material or therapeutic composition. Osteogenic materials include, without limitation, autograft, allograft, xenograft, demineralized bone, synthetic and natural bone graft substitutes, such as bioceramics and polymers, and osteoinductive factors. A separate carrier to hold materials within the device may also be used. These carriers may include collagen-based carriers, bioceramic materials, such as BIOGLASS®, hydroxyapatite and calcium phosphate compositions. The carrier material may be provided in the form of a sponge, a block, folded sheet, putty, paste, graft material or other suitable form. The osteogenic compositions may include an effective amount of a bone morphogenetic protein (BMP), transforming growth factor β1, insulin-like growth factor, platelet-derived growth factor, fibroblast growth factor, LIM mineralization protein (LMP), and combinations thereof or other therapeutic or infection resistant agents, separately or held within a suitable carrier material.

Embodiments of the invention may be applied to the lumbar spinal region, and embodiments may also be applied to the cervical or thoracic spine or between other skeletal structures. Some embodiments may also include supplemental fixation devices in addition to or as part of the devices disclosed herein to further supplement or replace spinal structures. For example, and without limitation, rod and screw fixation systems, anterior, posterior, or lateral plating systems, facet stabilization systems, spinal process stabilization systems, and any devices that supplement stabilization or replace spinal structures may be used as a part of or in combination with the devices.

An embodiment of the invention is a method of irradiating cells in or near a spinal structure. By way of non-limiting example, the spinal structure may be a portion of a vertebral column as depicted by vertebrae V1, V2 in FIG. 1. The method may include providing a spinal implant core, such as the spinal implant core 10 or 210, in which a radiation source, such as the radiation emitting device 1000, is substantially enclosed. The spinal implant core may be configured to replace at least a portion of the spinal structure. The spinal implant core may also include one or more radiolucent areas and one or more areas comprising a material that substantially blocks the transmission of radiation. Method embodiments may also include providing a shield, such as the shields 20, 120, 220, coupled to the spinal implant core that may be moved relative to the spinal implant core. Shields of some embodiments may also include one or more radiolucent areas and one or more areas comprising a material that substantially blocks the transmission of radiation

Method embodiments may include the act of implanting the spinal implant core, the radiation source, and the shield into a spinal structure between vertebrae to reinforce the spinal structure. The term “reinforce” as used herein may include acts of support of existing structures or the replacement of structures to serve in place of removed structures.

Some method embodiments may include the act of aligning at least one radiolucent area, such as one of the openings 17, 18, 19, 216, of the spinal implant core with at least one radiolucent area, such as one of the openings 27, 121-125, 229, of the shield. Such an alignment act may allow radiation to be therapeutically delivered to cells in or near the spinal structure. Therapeutically effective locations may include locations where a tumor or cancerous cells are present or suspected to be present, or areas from which a tumor or cancerous growth has been surgically removed. Therapeutically effective locations may also include areas where tissue growth is to be retarded, such as but not limited to, typical areas of scar tissue growth. The act of aligning radiolucent areas to allow radiation delivery may be accomplished prior to implanting one or more of the spinal implant core, the radiation source, and the shield into a patient. In other words, a device may be implanted in a configuration that emits radiation or in a configuration where radiation may not be emitted without further alignment of a shield relative to a spinal implant core or another further act.

In some embodiments, each radiolucent area of a spinal implant core is aligned with an area of a shield that comprises a material that substantially blocks the transmission of radiation, and each radiolucent area of the shield is aligned with an area of the spinal implant core that comprises a material that substantially blocks the transmission of radiation. By this act, radiation is prevented from being therapeutically delivered to cells in or near the spinal structure. In some method embodiments, the transmission of radiation to cells in or near the spinal structure may be selectively controlled by selectively aligning radiolucent areas and areas that substantially block the transmission of radiation of one component with radiolucent areas and areas that substantially block the transmission of radiation of another component. Through various control and diagnostic mechanisms, a treatment plan may be implemented by these varied alignments.

The radiation source or components of the radiation source may be inserted one or more of pre-operatively, inter-operatively, and post-operatively. The radiation source, for example the radiation emitting device 1000, may be a device capable of receiving radiation or components that emit radiation and may not at all times be able to emit radiation. That is, its designation as a “radiation source” does not mean that it, or one or more of its component parts, are at all times capable of emitting radiation.

Embodiments of the device for supplementing or replacing a spinal structure and therapeutically delivering radiation may be implanted from any surgical approach, including but not limited to, posterior, lateral, anterior, transpedicular, lateral extracavitary, in conjunction with a laminectomy, in conjunction with a costotransversectomy, or by any combination of these and other approaches.

Various method embodiments of the invention are described herein with reference to particular devices. However, in some circumstances, each disclosed method embodiment may be applicable to each of the devices, or to some other device operable as disclosed with regard to the various method embodiments.

Terms such as anterior, posterior, lateral, side, within, top, bottom, inside, outside, and the like have been used herein to note relative positions. However, such terms are not limited to specific coordinate orientations, but are used to describe relative positions referencing particular embodiments. Such terms are not generally limiting to the scope of the claims made herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. A device for supplementing or replacing a spinal structure and therapeutically delivering radiation to tissue within or near the spinal structure comprising:
a spinal implant core configured to be placed between a first vertebra and a second vertebra to supplement or replace at least a portion of the spinal structure, wherein the spinal implant core has at least one wall with a length between a first end and a second end, and wherein the spinal implant core includes one or more radiolucent areas and one or more areas comprising a material that substantially blocks the transmission of radiation;
a radiation source located within the spinal implant core and configured to deliver radiation; and
a shield coupled to the spinal implant core that is movable relative to the spinal implant core, wherein the shield has a length between a first end and a second end, wherein the shield includes one or more radiolucent areas that may be aligned with the one or more radiolucent areas of the spinal implant core to selectively permit radiation to be therapeutically delivered to tissue within or near the spinal structure.

2. The device of claim 1 wherein the device is a vertebral body replacement implant.

3. The device of claim 1 wherein the one or more radiolucent areas of the spinal implant core are areas where material has been removed to provide openings.

4. The device of claim 1 wherein the one or more radiolucent areas of the spinal implant core are closed areas comprising radiolucent material.

5. The device of claim 1 wherein the one or more radiolucent areas of the shield are areas where material has been removed to provide openings.

6. The device of claim 1 wherein the one or more radiolucent areas of the shield are closed areas comprising radiolucent material.

7. The device of claim 1 wherein the shield is coupled to the spinal implant core on an outside surface of the spinal implant core at least one wall.

8. The device of claim 1 wherein the length of the shield is less than the length of the spinal implant core and the spinal implant core is configured to contact a first vertebra at the first end of the spinal implant core and the second vertebra at the second end of the spinal implant core.

9. The device of claim 8 wherein the shield is movable along the length of the spinal implant core to align and to misalign one or more radiolucent areas of the spinal implant core with one or more radiolucent areas of the shield.

10. The device of claim 1 wherein the shield includes two or more radiolucent areas that may be alternatively aligned with one radiolucent area in the spinal implant core in different states of rotation by rotating the shield relative to the spinal implant core about a common axis with the spinal implant core.

11. The device of claim 1, further comprising an actuator for enabling movement between the spinal implant core and the shield.

12. A device for supplementing or replacing a spinal structure between a first vertebra and a second vertebra and for therapeutically delivering radiation to tissue within or near the spinal structure comprising:
a first tubular member having at least one wall with a length between a first end and a second end, wherein the first end contacts the first vertebra and the second end contacts the second vertebra, and wherein the first tubular member includes one or more radiolucent areas and one or more areas comprising a material that substantially blocks the transmission of radiation; and
a second tubular member coupled to the first tubular member and having at least one wall with a length between a first end and a second end, wherein the second tubular member includes one or more radiolucent areas and one or more areas comprising a material that substantially blocks the transmission of radiation, and wherein the second tubular member is movable relative to the first member to control alignment of radiolucent areas of the first tubular member and radiolucent areas of the second tubular member;
wherein the first tubular member and the second tubular member are configured to receive a radiation source.

13. The device of claim 12 wherein the device is a vertebral body replacement implant.

14. The device of claim 12 wherein the one or more radiolucent areas of the first tubular member are areas where material has been removed to provide openings.

15. The device of claim 12 wherein the one or more radiolucent areas of the first tubular member are closed areas comprising radiolucent material.

16. The device of claim 12 wherein the one or more radiolucent areas of the second tubular member are areas where material has been removed to provide openings.

17. The device of claim 12 wherein the one or more radiolucent areas of the second tubular member are closed areas comprising radiolucent material.

18. The device of claim 12 wherein the second tubular member is coupled to the first tubular member on an outside surface of the first tubular member at least one wall.

19. The device of claim 12 wherein the second tubular member is coupled to the first tubular member on an inside surface of the first tubular member at least one wall.

20. The device of claim 12, further comprising an actuator for enabling movement between the first tubular member and the second tubular member.

21. The device of claim 12 wherein the first tubular member includes two or more radiolucent areas that may be alternatively aligned with one radiolucent area in the second tubular member in different states of rotation by rotating the shield relative to the first tubular member about a common axis with the second tubular member.

22. A method of irradiating cells in or near a spinal structure comprising:
providing a spinal implant core in which a radiation source is substantially enclosed, the spinal implant core being configured to replace at least a portion of the spinal structure, wherein the spinal implant core includes one or more radiolucent areas and one or more areas comprising a material that substantially blocks the transmission of radiation;
providing a shield coupled to the spinal implant core that is movable relative to the spinal implant core, wherein the shield includes one or more radiolucent areas and one or more areas comprising a material that substantially blocks the transmission of radiation;
implanting the spinal implant core, the radiation source, and the shield into a spinal structure between vertebrae to reinforce the spinal structure;
aligning at least one radiolucent area of the spinal implant core with at least one radiolucent area of the shield to allow radiation to be therapeutically delivered to cells in or near the spinal structure; and
aligning each radiolucent area of the spinal implant core with an area of the shield that comprises a material that substantially blocks the transmission of radiation and aligning each radiolucent area of the shield with an area of the spinal implant core that comprises a material that substantially blocks the transmission of radiation such that radiation is not therapeutically delivered to cells in or near the spinal structure.

23. The method of claim 22 wherein the act of aligning at least one radiolucent area of the spinal implant core with at least one radiolucent area of the shield is accomplished prior to the act of implanting the spinal implant core, the radiation source, and the shield.

24. The method of claim 22 wherein the act of aligning at least one radiolucent area of the spinal implant core with at least one radiolucent area of the shield is accomplished after the act of implanting the spinal implant core, the radiation source, and the shield.
